(19) United States
(12) Patent Application Publication
(10) Pub. No.: US 2011/0151396 A1
(43) Pub. Date: Jun. 23, 2011

(54) TONGUE ISOLATOR EQUIPPED WITH LIGHTING

(76) Inventor: Yan-Zhang Lin, Puli Township (TW)

(21) Appl. No.: 12/640,008

(22) Filed: Dec. 17, 2009

(51) Int. Cl. A61B 1/247

(52) U.S. Cl. 433/31

(57) ABSTRACT

A tongue isolator equipped with lighting comprises a handgrip and an expanding portion which includes a connecting member and a first expanding wall and a second expanding wall that are connected to two sides of the connecting member to jointly form a U-shape with an opening facing forward. The handgrip has a front end connected to the first expanding wall. The first and second expanding walls are formed in an arched shape to mate the curvature of gum. The first and second expanding walls spaced from each other have an upper distance greater than a lower distance. The first and second expanding walls further have respectively a mirror located at an inner side and an outer side with. Each mirror contains at least one lighting element. The isolator thus can be coupled on a patient's teeth to separate the tongue and cheek and illuminate each tooth surface.

### FIELD OF THE INVENTION

The present invention relates to a dental tool and particularly to a tooth isolator that mates the curvature of gum to isolate patient's tongue and illuminate each tooth surface.

### BACKGROUND OF THE INVENTION

In the conventional dental clinic practice, a dentist often has to insert different tools into patient's oral cavity to do inspection and treatment. During inspecting patient's tooth, a tooth isolator usually is being used to separate patient's tongue or cheek from the tooth surface. The conventional tooth isolator, referring to FIG. 1, includes a handgrip 1 and an oral mirror 2 located at the front end of the handgrip 1. The handgrip 1 and oral mirror 2 usually are formed at a selected angle between them to expose tooth and tooth surface to allow the dentist to perform inspection and treatment of the tooth. However, the conventional tooth isolator generally cannot separate the tongue and cheek to expose the tooth at the same time. Moreover, external light usually cannot sufficiently illuminate every tooth surface in the oral cavity. Hence the dentist has to put on a head lamp with one hand holding the isolator and the other hand holding a treating tool. While treating the tooth at the inner side of the cheek, patient's tongue and cheek have to be separated, and sufficient illumination has to be provided to light each tooth surface in the oral cavity. In practice, the dentist often has to illuminate the patient's oral cavity with the head lamp and the dentist's body has to be bent in a desired gesture to enable the head lamp to light a targeted tooth surface in the oral cavity. Such a practice often hinders inspection and treatment of the tooth in the oral cavity.

Hence to improve dental equipment to solve the trouble of the delicate treatment and make overall treatment more efficient is a goal pursued by the Inventor.

### SUMMARY OF THE INVENTION

The primary object of the present invention is to provide a tongue isolator to separate patient's tongue and cheek to expose each tooth surface, and also provide a lighting element equipped on the tongue isolator to insert into patient's oral cavity to illuminate teeth to facilitate dentist's inspection or treatment of the teeth.

To achieve the foregoing object, the tongue isolator equipped with lighting according to the present invention comprises a handgrip and an expanding portion.

The expanding portion includes a connecting member and a first expanding wall and a second expanding wall connected to the two sides of the connecting member to jointly form a U-shape with an opening facing forwards. The connecting member and the first and second expanding walls have bottoms spaced to form a gap. The handgrip has a front end connected to the first expanding wall and tilted upwards. The first and second expanding walls are formed in an arched shape to mate the curvature of gum. An upper distance between the first and second expanding walls is greater than a lower distance therebetween. The second expanding wall has a front end tilted upwards from the lower side to the upper side to form an inclined arched end. The first and second expanding walls further have respectively a mirror located at an inner side and an outer side. Each mirror contains at least one lighting element.

Thus the expanding portion can mate the curvature of the gum to facilitate separation of the tongue and cheek to expose each tooth surface. The lighting element located on each mirror can illuminate each tooth surface to aid the dentist to inspect and treat the tooth in the oral cavity.

The foregoing, as well as additional objects, features and advantages of the invention will be more readily apparent from the following detailed description, which proceeds with reference to the accompanying embodiment and drawings. The embodiment serves merely for illustrative purpose and is not the limitation of the invention.

### BRIEF DESCRIPTION OF THE DRAWINGS

FIG. 1 is a perspective view of a conventional tooth isolator.

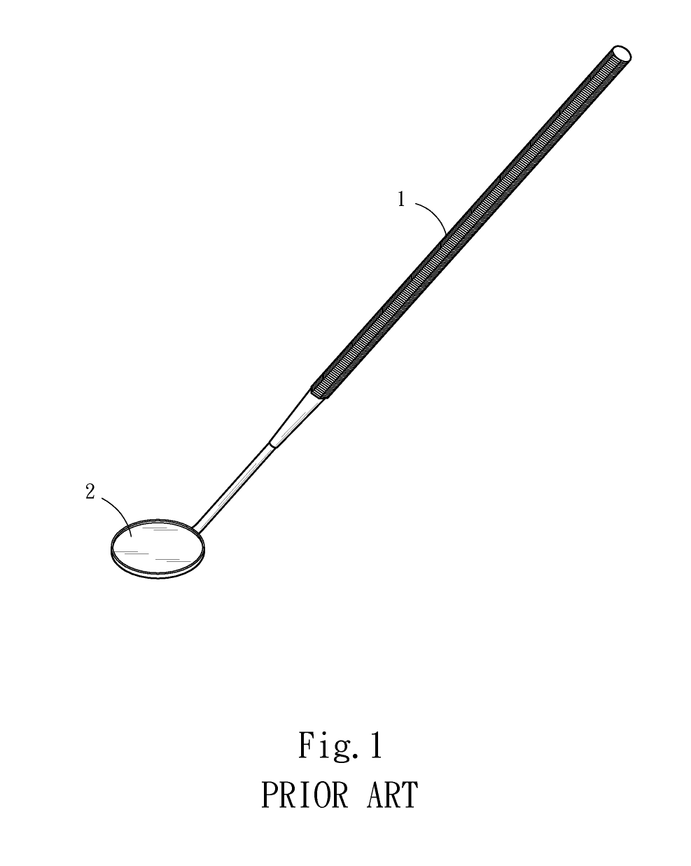

FIG. 2 is a perspective view of an embodiment of the present invention.

FIG. 3A is a top view of an embodiment of the present invention.

FIG. 3B is a cross-sectional view on line A-A in FIG. 3A.

FIG. 4 is a schematic view of an oral cavity.

FIG. 5 is a plane view of the present invention coupling on the teeth in the oral cavity.

FIG. 6 is a plane view of the present invention coupling on the tooth.

### DETAILED DESCRIPTION OF THE PREFERRED EMBODIMENT

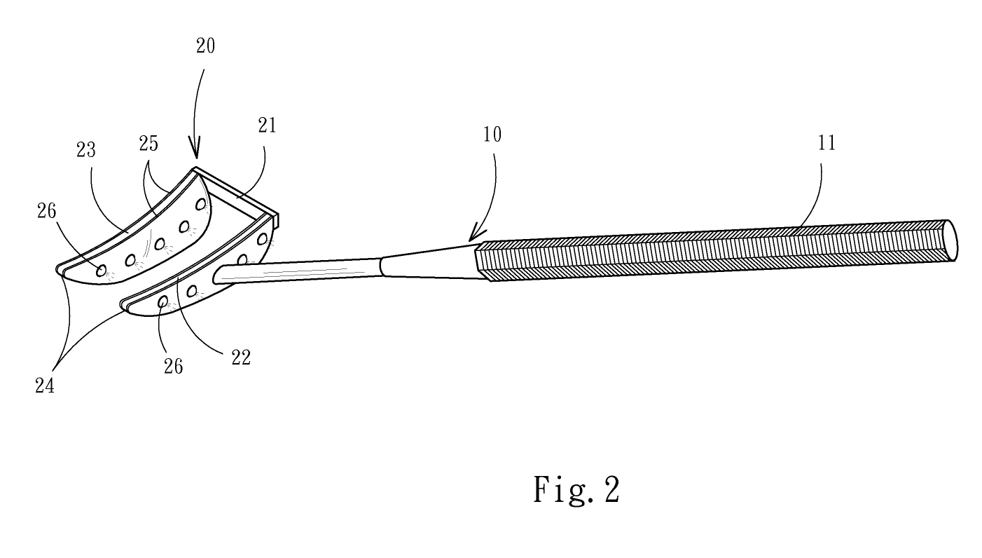

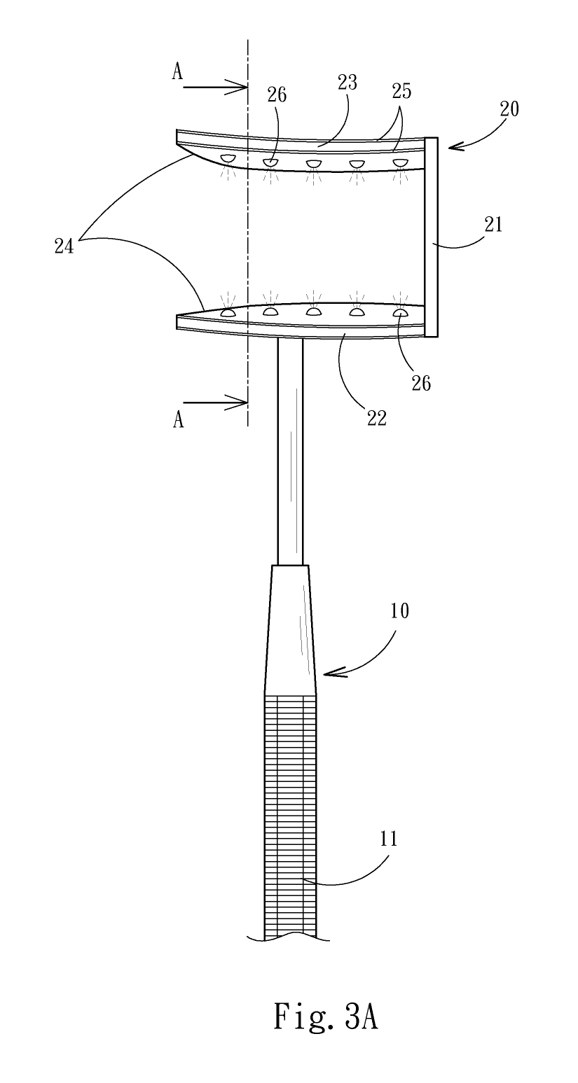

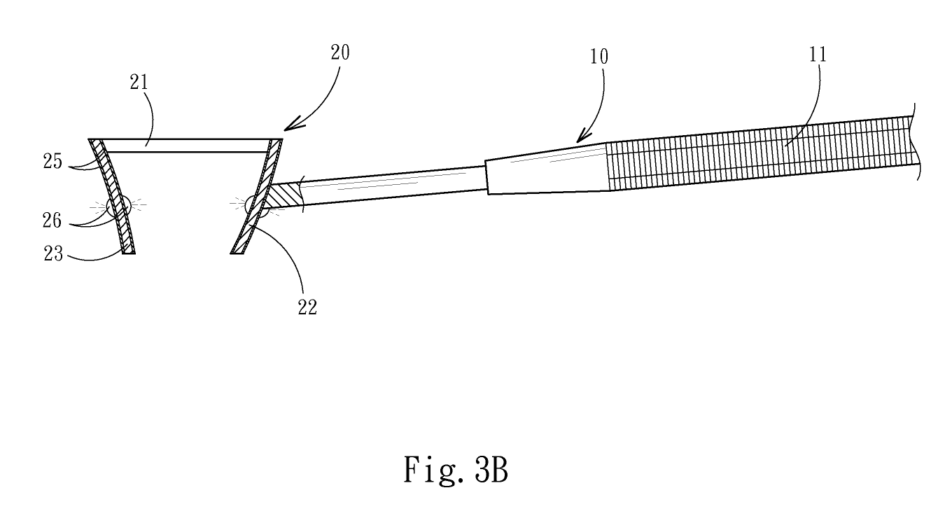

Please refer to FIGS. 2, 3A and 3B for an embodiment of the present invention. The tongue isolator equipped with lighting according to the present invention comprises a handgrip 10 and an expanding portion 20. The handgrip 10 has a front end and a rear end. The front end is formed at a diameter smaller than that of the rear end. The rear end of the handgrip 10 has a plurality of annular anti-slipping traces 11 formed thereon.

The expanding portion 20 includes a connecting member 21 and a first expanding wall 22 and a second expanding wall 23 that are connected to the two sides of the connecting member 21 to jointly form a U-shape with an opening facing forward. The connecting member 21 and the first and second expanding walls 22 and 23 have bottoms formed a gap between them. The front end of the handgrip 10 is connected to an outer side of the first expanding wall 22 and tilted upwards. The first and second expanding walls 22 and 23 are formed in an arched shape mating the curvature of gum. An upper distance between the first and second expanding walls 22 and 23 is greater than a lower distance therebetween. The first and second expanding walls 22 and 23 further have respectively a front end tilted upwards from the lower side towards the upper side to form an inclined arched end 24.

The first and second expanding walls 22 and 23 further have respectively a mirror 25 on an inner side and an outer side. Each mirror 25 has at least one lighting element 26 located thereon (the lighting element 26 of the present invention is a light emitting diode (LED), and each mirror 25 has a plurality of LEDs located thereon).

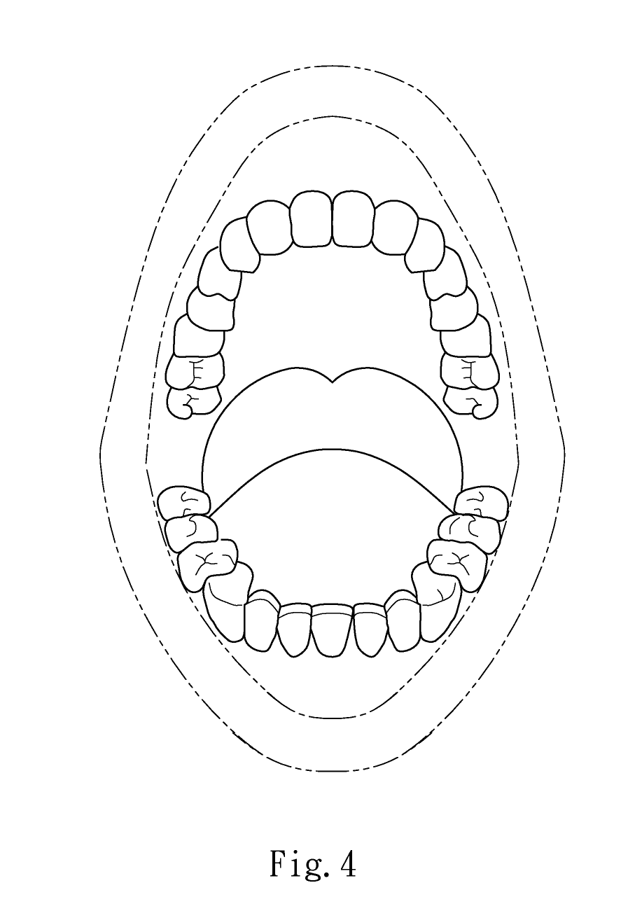

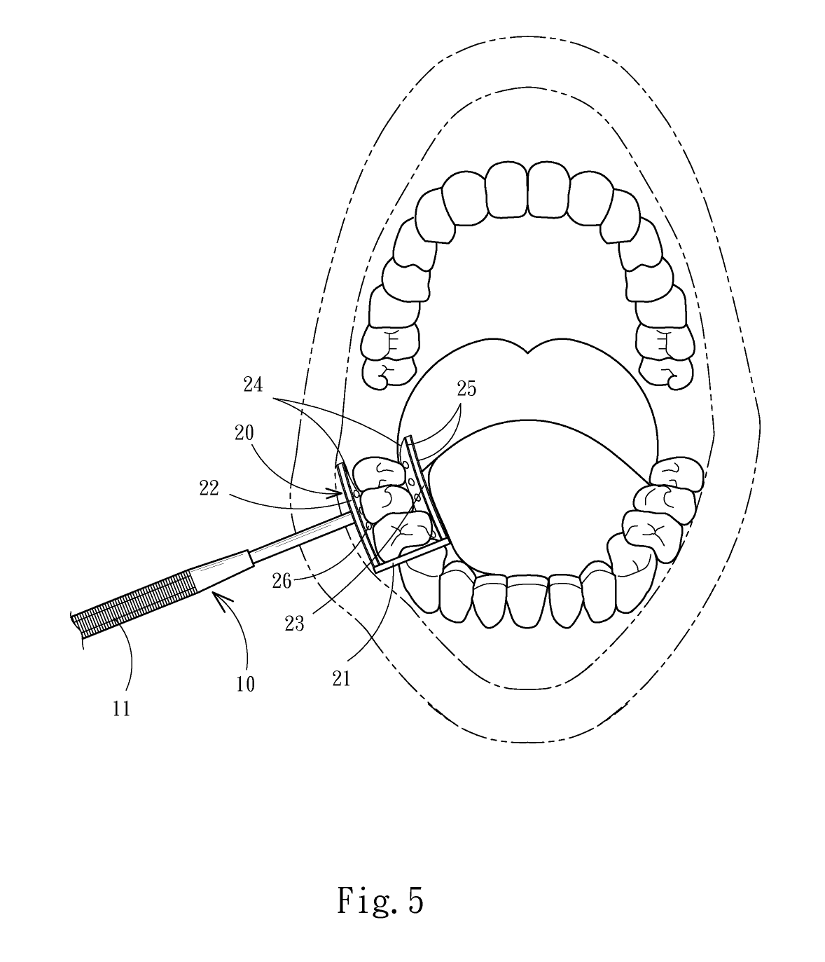

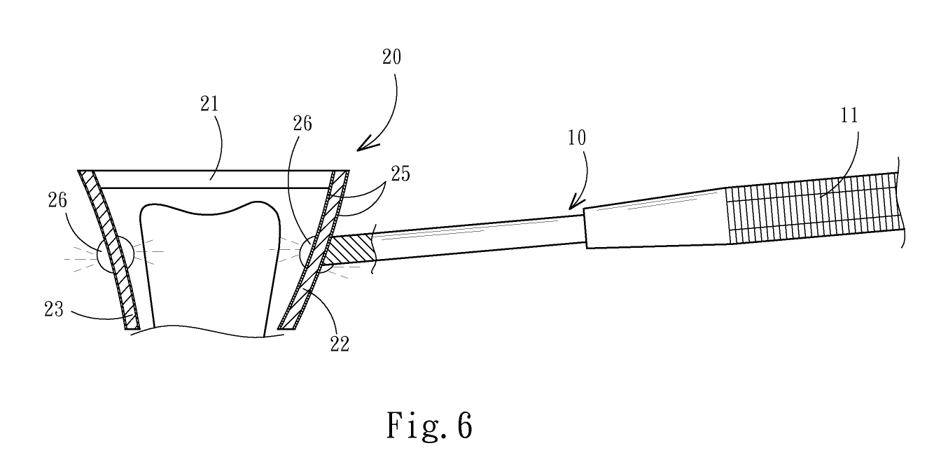

When in use, referring to FIGS. 4 through 6, the dentist holds the handgrip 10 on the anti-slipping traces 11 and inserts the expanding portion 20 into the mouth of a patient. The first and second expanding walls 22 and 23 can mate the curvature of patient's gum to hold teeth frame between them. The connecting member 21 is located above the tooth surface. As the upper distance between the first and second expanding walls 22 and 23 is greater than the lower distance therebetween, patient's tongue and cheek can be expanded to expose each teeth surface. Since the front ends of the first and second expanding walls 22 and 23 are tilted upwards from the lower side to the upper side to form the inclined arched end 24, the arched end 24 is formed in a profile to facilitate insertion of the expanding portion 20 into the patient's mouth to couple on the teeth.

With the mirror 25 located respectively on the inner side and outer side of the first and second expanding walls 22 and 23, and the lighting element 26 on each mirror 25 can emit light, each tooth surface can be illuminated at a shorter distance to make dentist's inspection and treatment of patient's teeth easier.

By means of the foregoing structure, the present invention provides many advantages, notably:

The first and second expanding walls 22 and 23 can separate patient's tongue and cheek at the same time to expose each tooth surface. Each mirror 25 can illuminate the tooth and the tooth can be lighted at a shorter distance through the lighting element 26 of the mirror 25. Therefore, it helps the dentist to inspect and treat patient's teeth. It also can be used in daily life to inspect teeth and clear dregs stuffed in the crevices of teeth of aged people or children without holding a flashlight with one hand. Thus it eliminates the problems of the conventional technique that is difficult to hold simultaneously the tongue isolator, lighting fixture and tools used to clean the dregs from the teeth.

What is claimed is:

1. A tongue isolator equipped with lighting, comprising:
a handgrip which includes a front end and a rear end; and
an expanding portion which includes a connecting member, a first expanding wall and a second expanding wall that are connected to two sides of the connecting member to jointly form an opening facing forwards, bottoms of the connecting member and the first and second expanding walls formed a gap therebetween, the front end of the handgrip being connected to an outer side of the first expanding wall, the first expanding wall and the second expanding wall being formed in an arched shape mating the curvature of gum;
wherein the first and second expanding walls include respectively a mirror on an inner side and an outer side thereof, each mirror containing at least one lighting element.

2. The tongue isolator of claim 1, wherein the lighting element is a light emitting diode.

3. The tongue isolator of claim 2, wherein the mirror includes a plurality of light emitting diodes located thereon.

4. The tongue isolator of claim 1, wherein the first expanding wall and the second expanding wall which are spaced from each other includes an upper distance greater than an lower distance.

5. The tongue isolator of claim 1, wherein the first expanding wall and the second expanding wall include a front end which is tilted upwards from a lower side towards an upper side to form an inclined arched end.

6. The tongue isolator of claim 1, wherein the handgrip is tilted upwards from the outer side of the first expanding wall.

7. The tongue isolator of claim 1, wherein the front end of the handgrip is formed at a diameter smaller than that of the rear end.

8. The tongue isolator of claim 1, wherein the rear end of the handgrip contains a plurality of annular anti-slipping traces.
